(19) United States
(12) Patent Application Publication
(10) Pub. No.: US 2005/0069937 A1
(43) Pub. Date: Mar. 31, 2005

(54) Spotting method onto and data reading method from biochemical analysis unit

(75) Inventors: Nobuhiko Ogura, Kanagawa (JP); Masashi Hakamata, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: Fuji Photo Film Co., Ltd.

(21) Appl. No.: 10/951,814

(22) Filed: Sep. 29, 2004

(30) Foreign Application Priority Data
Sep. 30, 2003 (JP) 2003-342626

(51) Int. Cl. C12Q 1/68; A61L 2/00; C12M 1/34

(52) U.S. Cl. 435/6; 435/287.2; 427/2.11

(57) ABSTRACT

In a biochemical analysis unit, a membrane is pressed into through holes formed in a substrate to form plural spot areas in each through hole, and the remaining membrane forms a thin layer on a rear face of the substrate. A probe solution is spotted on each spot area by a spot head. On the occasion of the spotting, a pin arrangement plate presses the biochemical analysis unit from the rear face. On the pin arrangement plate, pins are arranged at the same pitch as the spot areas. The pin arrangement plate is positioned such that the pins confront the corresponding spot areas. When the pressing is made, the pins push the thin layer to enter the corresponding through holes. Thus the pores in the thin layer are squashed, and it is prevented that the probe solution penetrate into the neighboring spot areas.

### BACKGROUND OF THE INVENTION

1. Field of the Invention

The present invention relates to a spotting method onto and a data reading method from a biochemical analysis unit.

2. Description Related to the Prior Art

In order to make a biochemical analysis for base sequence of substances derived from living organism (for example DNA), a biochemical analysis unit is used. In order to obtain the biochemical analysis unit, minute through holes are formed in the substrate, and porous materials and the like are pressed into each through hole to form a spot area. Thus, the spot areas are arranged on the substrate, and therefore the biochemical analysis unit is called also microarray. A method of biochemical analysis, in which the biochemical analysis unit is used, includes a spotting process, a reaction process, a data reading process, and a data analysis process. In the spotting process, a specific binding substance as a reagent (hereinafter probe) is spotted and fixed in the spot areas on the biochemical analysis unit. In the reaction process, a specific binding substance as a test body (hereinafter target) is penetrated into the spot areas, and the specific binding (the biding between the probe and the target) is made. In the data reading process a biochemical analysis data is read out from the biochemical analysis unit as a result of the specific binding reaction in each spot area. In the data analysis process, the read out analysis data is analyzed in the personal computer and the like. (see, Japanese Patent Laid-Open Publication No. 2003-215125).

Since the probe is a reagent for searching the information of expression, the molecular structure (for example base sequence, composition and the like) of the used probe is already known. As the probe, there are hormones, tumor markers, enzymes, antibodies, antigens, abzymes, receptors, other proteins, ligand, nucleic acids, cDNA, DNA, RNA, and the like, and the probe can make a specific binding to the target, whose molecular structure is not known. As the target, there are substances derived from living organism (such as hormones, tumor markers, enzymes, antibodies, antigens, abzymes, receptors, other proteins, ligand, nucleic acids, cDNA, DNA, mRNA, and the like, which are extracted and isolated from the living organism), and products obtained by performing the chemical treatments or the chemical modifications of the substances derived from living organism.

When the base sequence is searched, several sorts of the probes are fixed in respective spot areas of the biochemical analysis unit. Then in the reaction process, a solution in which the target is dissolved to a solvent is penetrated in the spot areas, and the specific binding of the target and the probe having a complementary relation to the target is made.

In order to detect the specific binding, the reaction solution contains for example a labeling substances. As the labeling substances to be used, there are fluorescent substances which generate a chemical fluorescence in a chemical reaction. After the specific binding is made, the biochemical analysis unit is cleaned to remove the reaction solution on other areas than spot areas.

In the spot area in which the specific binding is made, the labeling substances remain. Accordingly, in the data reading process, a labeling signal of the optical ray, the radioactive ray or the like generated from the labeling substances is read for detecting the specific binding. As the detecting device to be used, there is an imaging device, for example, a CCD imaging sensor for reading the optical information.

In order to form the spot area of the biochemical analysis unit, the adsorptive material is pressed onto a rear face of the substrate so as to extend thereon, and the adsorptive material is supplied into the through holes. Thereby, as part of the adsorptive material remains to form a thin layer on the rear face of the substrate, and the adsorptive materials in the neighboring through holes are connected through the thin layer on the rear face. Accordingly, the labeling material remaining in the spot area sometimes penetrates through the thin layer into the neighboring spot areas. In this case, the labeling substance spotted in one spot area generates the light also in the neighboring spot areas. Thus the light seepage occurs, and is observed as a data noise which lowers the accuracy of the data. In order to obtain the data with high accuracy, it is necessary to detect the accurate light intensity of each spot, and namely to reduce the light seepage.

Accordingly, in the publication No. 2003-215125, a light absorptive material is contained in the thin layer between the spot areas, so as to reduce the light seepage.

However, in this method in which the light absorptive material is used, the effect of reducing the light seepage is not enough. Therefore, in order to obtain the analysis data of higher accuracy, the more effective method is required.

### SUMMARY OF THE INVENTION

An object of the present invention is to provide a spotting method onto and a data reading method from a biochemical analysis unit, in which the noise caused by a light seepage is reduced and the data reading is possible with high accuracy.

In order to achieve the above object and other objects, in a spotting method for fixing a probe in plural spot areas formed in a biochemical analysis unit which includes a substrate and a membrane disposed on a back of the substrate, plural holes are arranged at a predetermined pitch in the substrate, and the membrane has adsorption properties. The each spot area is constructed of the through hole and a part of the membrane charged into the through hole. In the spotting method, plural pins are pressed on a back of the membrane, and the plural pins are disposed at the predetermined pitch corresponding to the plural through holes. Then a liquid of the probe is spotted into said spot areas with a spot head from an opposite side to the pin. Note that it is preferable to spot the liquid with the substrate pressed on the pin arrangement plate. In this case, the substrate is pressed on the front surface by a contact member, which is provided for the spot head to contact around the spot area.

In a data reading method for reading a reaction result in plural spot areas formed in a biochemical analysis unit which includes a substrate and a membrane disposed on a back of the substrate, plural holes are arranged at a predetermined pitch in the substrate, and the membrane has adsorption properties. The each spot area is constructed of the through holes and a part of the membrane charged into the through holes. A probe is previously fixed in the each spot area. In the data reading method, a reaction solution containing a target as a test body passes through the spot area, and the target makes a specific binding to the probe. The data reaction result in each spot area is optically read from an opposite side to the pin.

In the present invention, the biochemical analysis unit is confronted to the pin arrangement plate, and pressed to the pin arrangement plate such that the pins of the pin arrangement plate may push the thin layer of the membrane into the corresponding through holes formed in the substrate of the biochemical analysis unit. Thereby the reaction solution is spotted on the spot area formed of the membrane in each through hole, and otherwise the result of the reaction is read as the reaction data. Therefore, the probe solution does not penetrate into the neighboring spot areas and the light from the one spot area does not mixed to that from the neighboring spot areas. Accordingly, the light seepage is prevented and the data reading is made with high accuracy.

### BRIEF DESCRIPTION OF THE DRAWINGS

The above objects and advantages of the present invention will become easily understood by one of ordinary skill in the art when the following detailed description would be read in connection with the accompanying drawings.

FIG. 1 is a flow chart illustrating all processes of biochemical analyzing method in which biochemical analysis unit is used;

FIG. 2A is a sectional side view of a substrate;

FIG. 2B is a flow chart of procedure of production of the biochemical analysis unit;

FIG. 2C is an explanatory view of producing the biochemical analysis unit;

FIG. 2D is a sectional view of the biochemical analysis unit;

FIG. 3A is a plan view of a biochemical analysis unit;

FIG. 3B is an exploded plan view of a biochemical analysis unit;

FIG. 4A is an explanatory view of the situation before the spotting;

FIG. 4B is an explanatory view of the situation after the spotting;

FIG. 5 is a perspective view illustrating a positional relation between the biochemical analysis unit and a pin arrangement plate;

FIG. 6 is a flow chart of an indirect labeling method;

FIG. 7 is an explanatory view illustrating an embodiment of a data reading;

FIG. 8 is an explanatory view illustrating another embodiment of a data reading.

### PREFERRED EMBODIMENTS OF THE INVENTION

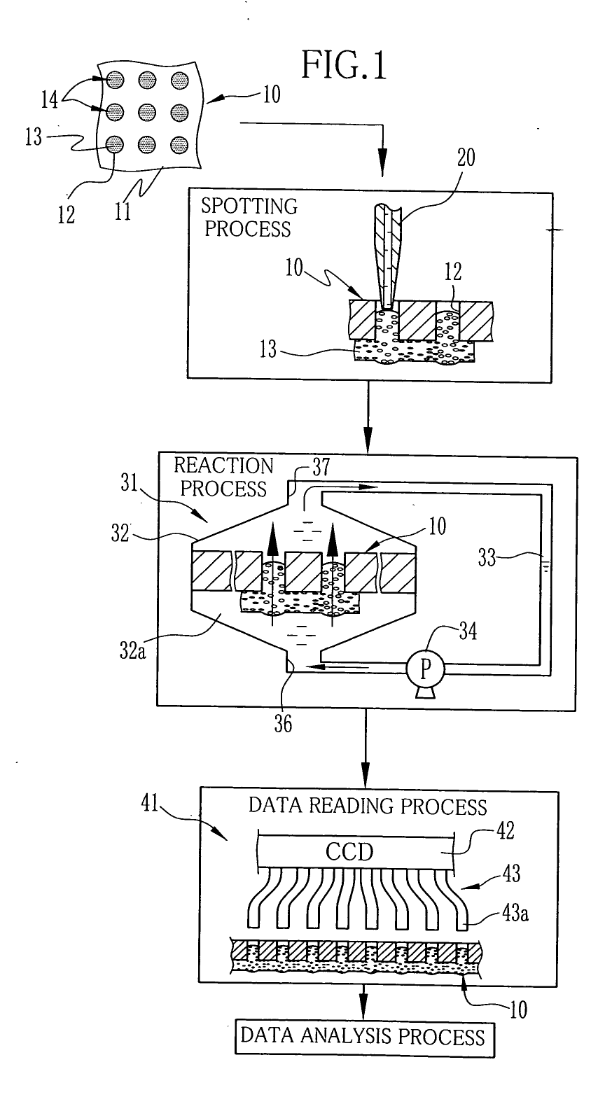

As shown in FIG. 1, a biochemical analyzing method in which a biochemical analysis unit 10 is used includes a spotting process, a reaction process, a data reading process and a data analysis process. In the biochemical analysis unit 10, minute through holes 12 are formed in matrix-arrangement in the substrate 11, and a membrane 13 of the adsorptive material is pressed into the through holes 12. Thus a spot area 14 are formed in each through hole 12, and the obtained biochemical analysis unit is a flow-through type.

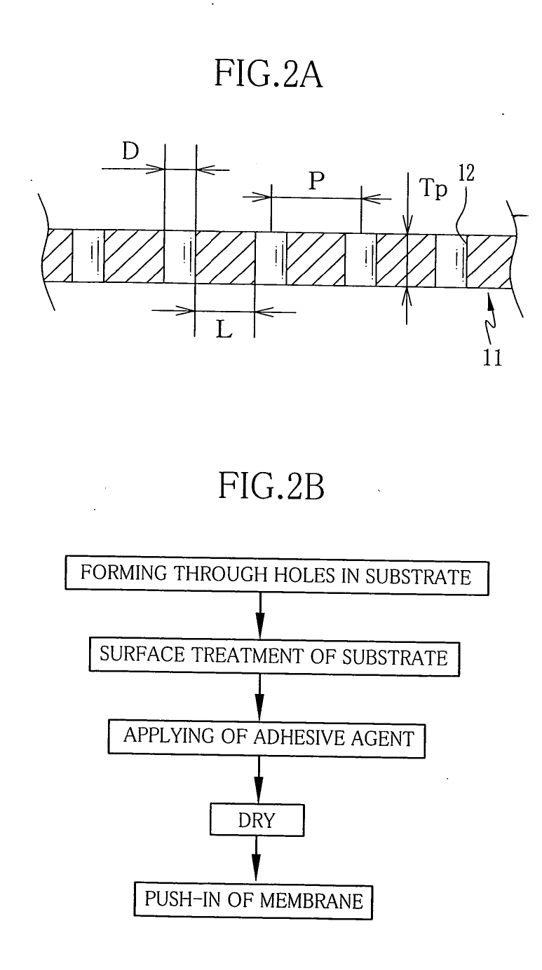

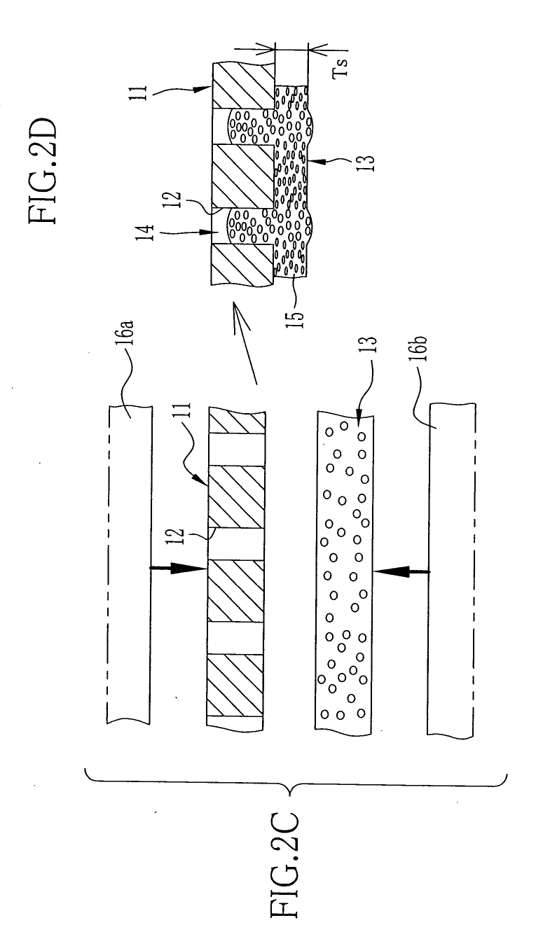

FIGS. 2A-2D are explanatory views of a method of producing the biochemical analysis unit 10. The substrate 11 is formed of the materials which can decrease the light intensity so as to prevent the generation of the light toward the neighboring spot areas, for example metal, ceramics, plastics, and the like. When the light does not pass toward the neighboring spot areas, it is prevented to misunderstand that the light would be generated from the other areas than the some spot areas from which the light is generated. When the materials having high effect for decreasing the light intensity is used, the misunderstanding is prevented, and the analysis data having high reliability is obtained. The rate of decreasing the intensity of the light generated from the one spot area becomes preferably at most ⅕, and especially at most 1/10 in the neighboring spot area.

As shown in FIG. 2A, the thickness Tp of the substrate is preferably in the range of 50 to 1000 μm, and especially in the range of 100 to 500 μm. As the metals, there are copper, silver, gold, zinc, plumbum, aluminum, titanium, tin, chromium, iron, nickel, cobalt, tantalum and the like. Further, alloys, such as stainless, brass and the like, may be used. However, the metals are not restricted in them. Furthermore, as the ceramics, there are alumina, zirconia and the like. However, the materials to be used are not restricted in them.

As the plastics, there are olefins (for example, polyethylene, polypropylene, and the like), polystyrene, acryl resin (for example, polymethylmethacrylate, and the like), polymers containing chlorine (for example, polyvinyl chloride, polyvinylidene chloride and the like), polymers containing fluorine (for example, polychlorotrifluoroethylene, and the like), polycarbonates, polyesters, (for example, polyethylene naphthalate, polyethylene telephthalate and the like), polyamide (for example nylon-6, nylon-66 and the like), polyimide, polysulfonate, polyphenylen sulfide, silicon resins (for example, polydiphenyl cyclohexane and the like), phenol resins (for example, noborac and the like), epoxy resins, polyurethane, celluloses (for example, cellulose acetate, nitrocellulose and the like), and the like. Further, there are copolymers (for example butadiene-cellulose copolymer, and the like). Furthermore the above polymers may be blended. However, the sorts of the plastics are not restricted in them.

It is preferable to use the plastics as the materials of the substrate, since the through holes are easily formed. However, in this case, the light intensity is hardly decreased. In order to decrease the light intensity moreover, preferably, metal oxide particles or glass fiber particles are added to the plastics, and dispersed therein. As the metal oxide particles, there are silicon dioxide, alumina, titanium dioxide, iron oxide, cupper oxide and the like. However, the sorts of the metal oxide are not restricted in them.

A method of forming the through holes 12 may be a punching method, a pulse discharging method, an etching method, and methods in which a laser beam (exima laser and YAG laser) is applied to the substrate. However, the method of forming the through holes is not restricted in them, and selectable depending on the material of the substrate.

In order to make the density of the through holes 12 higher, the area of a opening of the each through hole is preferably less than 5 mm², particularly less than 1 mm², and especially less than 0.3 mm², more especially less than 0.01 mm², and most especially less than 0.001 mm². Further, when the through hole has a nearly circular shape, its diameter is preferably from 200 μm to 300 μm.

A arrangement pitch P of the through holes 12 (a distance of centers between neighboring through holes 12) is preferably 50 μm to 3000 μm, and a length L of the nearest edges between the neighboring through holes is preferably 10 μm to 1500 μm. Further, the number of the through holes 12 in a unit area is preferably at least 10/cm², particularly at least 100/cm², especially at least 500/cm², most especially 1000/cm² to 10000/cm².

FIG. 2B shows a producing process of the biochemical analysis unit. In order to make the cleaning effect higher, a surface treatment is made on the substrate in which the through holes 12 are formed. When metals and alloys (for example stainless and the like) are used as the materials of the substrate 11, the surface treatment is made in at least one of corona discharging method, plasma discharging method and an anodic oxidization method. In the surface treatment, a surface treatment layer is formed on the substrate 11, and is a layer of metal oxide having hydrophilic property since it contains carbonyl groups and carboxyl groups.

After the surface treatment, an adhesive agent is applied to a surface of the substrate 11, onto which the membrane 13 is pressed for the insertion into the through holes 12. The method of applying the adhesive agent is not restricted. However, it can be a roller coating, a wire bar coating, a dip coating, a blade coating, an air knife coating or the like. As the adhesive agent, there are styrene-butadiene rubber and acrylonitril-butadiene rubber. However, it is not restricted in them. Note that the excess adhesive agent is scratched and removed by the blade, or may be removed with use of a laser beam for preventing the generation of the impurities in the following process. Note that the processes of the surface treatment of the substrate and the application of the adhesive agent can be omitted.

After the application of the adhesive agent, the membrane 13 is pressed into the through holes 12. As the membrane 13, there are porous materials and fiber materials. Note that the porous materials and the fiber materials are used simultaneously. The membrane 13 used in the present invention may be one of the porous materials (organic, inorganic porous materials or mixture thereof), the fiber materials (organic or inorganic fiber materials). Further these may be mixed. The thickness of the membrane 13 is not restricted especially. However, it may be in the range of 100 μm to 200 μm (0.10 mm to 0.20 mm). Further, a void ratio C in volume is preferably within 10% to 90% and pores forming the void would average 0.1 μm to 50 μm in diameter, and a void ratio C in volume is a percentage of a total volume of the void to the appearance volume of the adsorptive materials.

The sorts of the organic porous materials are not restricted especially. However, they are preferably polymers, for example, cellulose derivatives (for example, nitro cellulose, regenerated cellulose, cellulose acetate, cellulose acetate butyrate, and the like), aliphatic polyamides (for example, nylon-6, nylon-6,6, nylon 4,10, and the like), polyolefines (for example, polyethylene, polypropyrene), polymers containing chlorinate (for example, polyvinyl chloride, polyvinylidene chloride and the like), fluorine resins (for example, polyvinylidene floride, polytetrafluoride and the like), polycarbonate, polysulfone, alginic acid, and derivatives thereof (for example, calcium alginate, ion complex of alginic acid/polylysine, and the like) collagen, and the like. Further, the copolymer or the complexes (or mixture) of these polymers may be used. Note that porous nylon is preferably used in view of the water absorbing properties in the present invention.

The sorts of the inorganic porous materials are not restricted. However, they are preferably metal (for example, platinum, gold, iron, silver, nickel, aluminum, and the like), metal oxide (for example, alumina, silica, titania, zeolite, and the like), salts of metals (hydroxyapatite, calcium sulfate and the like) complexes of them, and the like. Further, porous carbon materials (activated carbon and the like) may be used.

Further, organic fiber materials and the inorganic fiber materials are not restricted especially. However, as the organic fiber materials, the cellulose derivatives, aliphatic polyamides and the like can be used, and as the inorganic fiber materials, glass fiber and metal fiber can be used. Note that in order to increase the strength of the membrane 13, insoluble fiber materials can be mixed with the solvent for the porous materials.

FIGS. 2C & 2D illustrate a method for pressing the membrane 13 into the through hole 12. A sheet of the membrane 13 is disposed on a rear face of the substrate 11, and the pressing is intermittently made from up and down sides by press plates 16a, 16b. Thus part of the membrane 13 is pressed into the through hole 12 to form the spot area 14, and another part is extended with the pressure to remain as a thin layer 15 on the rear face of the substrate 11. The thickness Ts of the thin layer 15 is about 30 μm-80 μm. If the organic porous or fiber materials are used as the membrane 13, the press plates are heated so as to increase the temperature of the substrate 11. Thus the membrane 13 becomes softened, and easily pressed into the through holes 12 to form the spot area 14. Further, a roller may be used instead of the press plates.

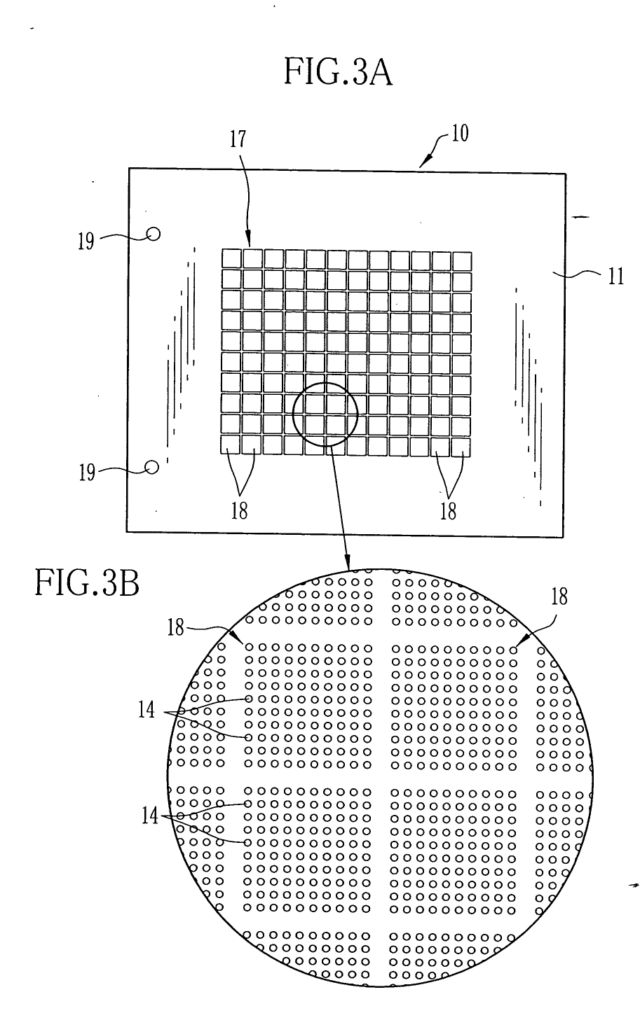

FIGS. 3A & 3B are plan views of the biochemical analysis unit 10. A flow-through area 17 is generally rectangle and is regularly sectioned into rectangle blocks 18. In each block 18, a predetermined number of the spot area 14 is arranged. The size of the substrate is, for example, 70 mm in length and 90 mm in width. The size of each block 18 is about 4 mm in every direction. The blocks 18 are matrix-likely arranged, for example, to 12 blocks in length, and 16 blocks in width. The features of the flow-through areas (namely size and the number of the block 18, and the size and the pitch of each spot area 14) are determined, corresponding to the feature of a detecting device. Positioning holes 19 are used for attachment of the biochemical analysis unit 10 to the reaction vessel 32. In this embodiment, the biochemical analysis unit 10 is sectioned into the blocks having the predetermined number of the spot areas 14. However this sectioning may not be made. For example, the spot areas 14 may be arranged all over the flow-through area 17 in equal pitch.

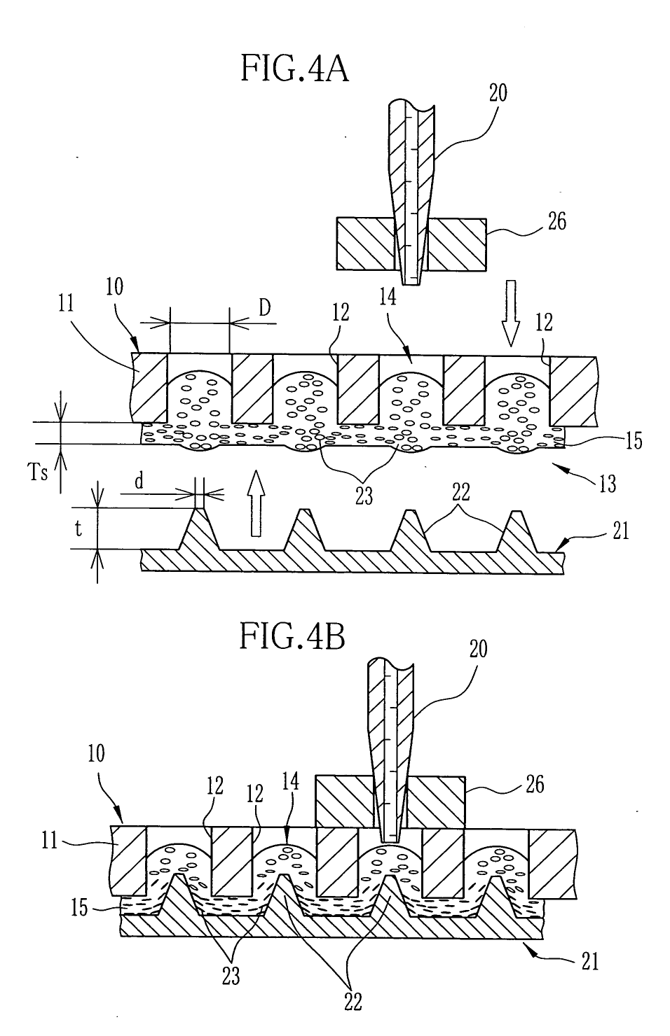

As shown in FIG. 4, in the spotting process, solutions containing various sorts of probes (hereinafter probe solutions) are spotted in the respective spot area 14 of the biochemical analysis unit 10 with use of a spotter. The spotter has a spot head, which has a groove on its top for spotting the prove solution. The plural sorts of probe solutions, which are dispensed on a well plate, are sucked up and spotted in each spot area 14 by the spot head 20.

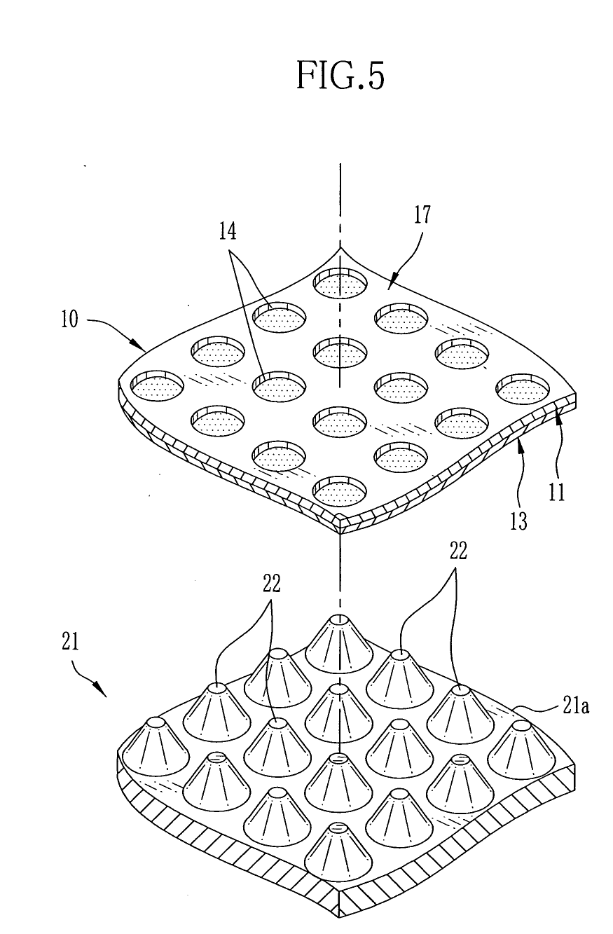

As shown in FIG. 4B, the spotting is made in the situation that a pin arrangement plate 21 presses a thin layer 15 from a rear side of the biochemical analysis unit 10. As shown in FIG. 5, the pin arrangement plate 21 has a base 21a and plural pins 22 formed in accordance with an arrangement pitch of the spot areas 14 of the biochemical analysis unit 10. Each pin 22 has a corn-like shape, and a height thereof from the base 21a is, for example, about 150 μm. A diameter d of a top of each pin 22 is smaller than a diameter D of the through hole 12 such that the top of the pin 22 can enter the through hole 12. Note that each pin 22 may have a pyramid-shape.

The pin arrangement plate 21 is disposed at such a position on a table of a spotter that each pin 22 may contact to a center of the corresponding spot area 14 (or the corresponding through hole 12). The biochemical analysis unit 10 is shifted downwards to the pin arrangement plate 21, and pressed onto the arrangement surface of the pins 22. Each pin 22, after contacting to the thin layer 15, enters the corresponding through hole 12 while the top of each pin 22 pushes part of the thin layer 15 into the through hole 12. Besides, since the thin layer 15 is pressed, pores 23 in the thin layer 15 are squashed. In this situation is spotted the probe solution onto the spot areas 14 from a front side of the substrate 11. In the lower side of the spot areas 14, since the pores 23 are squashed, the spotted probe solution does not interpenetrate through the thin layer 15 into the neighboring spot areas 14.

Concretely, the diameter d of the top of the pin 22 is determined in the range of a formula: D-2Ts≧d≧D/2. As described above, the diameter d of the top of the pin 22 is smaller than the diameter D of the through hole 12. However, if the diameter d is too small, a contact area of the top to the thin layer 15 becomes smaller, and the pore 23 cannot be squashed in a wide area. Thus the prevention effect to the interpenetration of the probe solution into the neighboring spot areas 14 is lost. The minimal value of the diameter d of the top is about ½ of the diameter D of the through hole 12. Further, since the pin 22 enters the through hole 12 with pressing the thin layer 15, it is necessary to keep at least a margin for the thickness Ts of the thin layer 15 between the pin 22 and an inner wall of the through hole 12. Viewing it in section, since the margin is needed for the thin layer 15 to enter both sides of the pin 22, the twice of the thickness Ts is necessary as the total margin in one radial direction. Accordingly, the maximal value of the diameter d is about D-2Ts. For example, if the diameter D of the through hole 12 is 300 μm and the thickness Ts of the thin layer 15 is 30 μm, the diameter d of the top of the pin 22 is determined in the range of 150 μm to 240 μn. Otherwise, if the diameter D of the through hole 12 is the same value of 300 μn and the thickness Ts of the thin layer 15 is 50 μm, the diameter d is determined in the range of 150 μm to 200 μm.

As shown in FIGS. 4A & 4B, a contact member (flange) 26 is provided for a top portion of the spot head 20. For example, the contact member 26 is attached to the spot head 20 such that they may move together. The position of the contact member 26 is designated, so as to contact to the surface of the substrate 11 when the descending spot head 20 arrives at a spotting position. The contact member 26 presses the substrate 11 toward the pin arrangement plate 21 in performance of the spotting. Therefore the thin layer 15 is sandwiched between the substrate 11 and the pin arrangement plate 21, and the pin arrangement plate 21 continuously presses the thin layer 15. Note in this embodiment that the substrate 11 is pressed with the contact member 26 attached to the spot head 20. However, a pressing device may be used for pressing the substrate 11 and the pin arrangement plate 21.

After spotting the probe solution, an UV-ray is irradiated on the spot areas 14 to fix the probe thereto.

In the reaction process, the specific binding reaction of the probes and a target as the test substance is made with use of a reactor 31. The reactor 31 is constructed of a reaction vessel 32, a circulating pipe 33 and a pump 34. In the reaction vessel 32, the biochemical analysis unit 10 is contained and a reaction solution 35 for performing the specific binding reaction is supplied.

The reaction solution is prepared by a preparing device for the reaction solution. In the preparing device, a target is dissolved in a solvent to prepare the reaction solution. The prepared reaction solution is contained in a tank (not shown) provided for the reactor 31. As the labeling materials, there are phosphor materials, radioactive materials, chemiluminescent materials which generate a chemiluminescence in the chemical reaction thereof, and the like. In this embodiment, the chemiluminescent materials are used. Further, introduced to perform the labeling is an indirect-labeling method, in which no labeling materials but antigens are contained in the reaction solution.

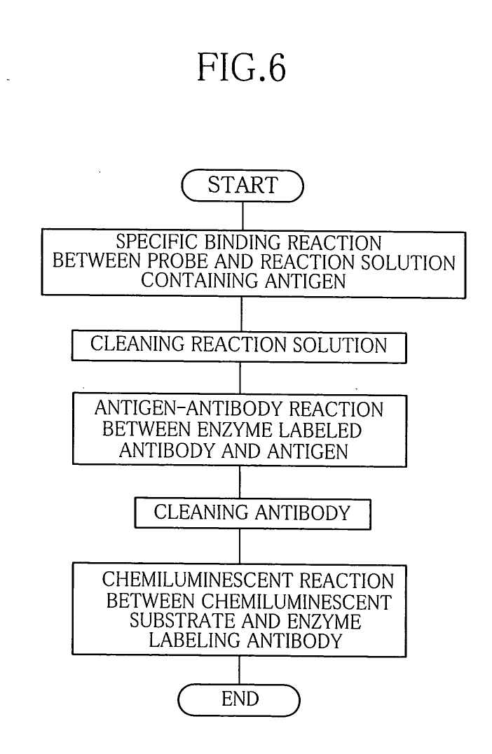

FIG. 6 is a flow chart illustrating processes of a reaction in the indirect labeling method with use of the above structure. At first, reaction solution containing antigens is supplied into the chamber so as to make a specific binding reaction to the probe. Thus in the spot area in which the specific biding reaction is made, the antigen remains. Thereafter, the cleaning is made with use of the cleaning solution to remove the reaction solution. Thereafter, supplied into the chamber is an antigen solution containing an enzyme-labeled antibody which makes the specific binding to the antigen. In the spot area in which the antigen remains, the enzyme labeled antibody which binds to the antigen will remain. Thereafter, the cleaning is made with use of an antibody cleaning solution, and thus the other enzyme labeled antibody which does not bind to the antigen is removed. After this cleaning, a chemiluminescent reaction of the enzyme labeling antibody to the chemiluminescent substrate as the labeling substance. As the labeling substance, for example, there are chemiluminescent substrates, such as CDP-star (trademark) and the like. The enzyme-labeling antibody decomposes the chemiluminescent substrate. In the decomposition, the chemiluminescent substrates generate a light.

The reaction vessel 32 is provided with an inlet 36 for supplying the reaction solution and an outlet 37 for discharging the reaction solution. The biochemical analysis unit 10 is set in the reaction vessel 32 such that one surface (the lower surface in this figure) may confront the inlet 36 and another surface (the upper surface in this figure) may confront the outlet 37. The reaction solution supplied into the reaction vessel 32 penetrates into each spot area 14 from the lower side in this figure. Then, the reaction solution passes through each spot area 14 and flows to the upper side in this figure. In some of the spot areas 14, in which the probes being complementary to the target are contained, the specific binding of the probes and the targets is made. Then the reaction solution flows through the spot areas 14 and is discharged through the outlet 37 from the reaction vessel 32.

The inlet 36 and the outlet 37 are connected to the circulating pipe 33, and the reaction solution discharged from the reaction vessel 32 is fed through the pump 34 and the circulating pipe 33 to the reaction vessel 32 again. Further, when the reaction solution or the cleaning solution is supplied into or discharged from the reaction vessel 32, a supply pipe (not shown) and a discharge pipe (not shown) are respectively connected to the inlet 36 and the outlet 37. The inlet 36 and the outlet 37 are exchangeably connected to the supply pipe, the discharge pipe and the circulating pipe 33.

The procedure described above for supplying into, discharging from a chamber 32a, and circulately feeding by a pump 34 is applied in the same manner to an antigen cleaning solution, an antigen-antibody reaction solution containing an enzyme labeling antigen, an antibody cleaning solution, and a solution containing chemiluminescent substrates.

After the reaction process, the biochemical analysis unit 10 is removed from the reactor 31 and sent to the data reading process. In the data reading process, the biochemical analysis data is photoelectrically read by a detecting device 41. Since the labeling substances remain in the some spot areas in which the specific binding reaction is made, the light is generated. Otherwise, the light is not generated in the other spot areas in which the specific binding is not made. Receiving the light as the result of the specific binding reaction in the spot areas 14, the detecting device 41 generates an image data. In the data analyzing process, the image data is analyzed as the biochemical analysis data.

The detecting device 41 includes a CCD image sensor 42 which receives a light generated from the labeling substances and photoelectrically converts the light. In front of a receiving surface of the CCD image sensor 42, there is a light guide for guiding the light to photosensitive elements of the CCD image sensor 42. A light guide 43 is constructed of optical fibers whose number is corresponding to that of the spot areas 14. One end of each optical fiber confronts the receiving surface and another end confronts the corresponding spot area 14.

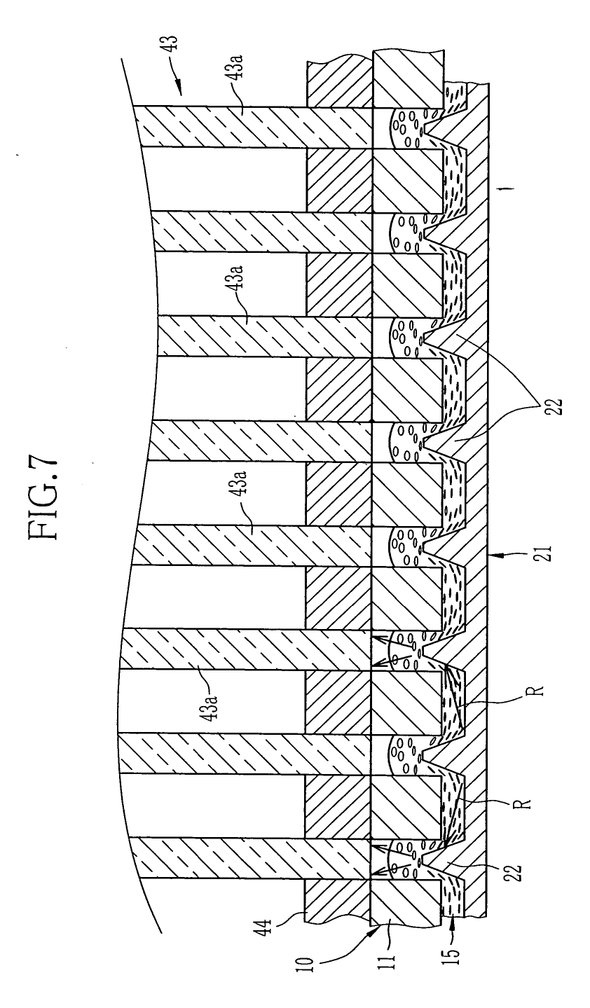

As shown in FIG. 7, the data reading is made in the same situation as the spotting, that the pin arrangement plate 21 contacts to the rear face of the biochemical analysis unit 10. At one end of the light guide 43, positioning member 44 is provided for positioning each optical fiber 43a to confront the corresponding spot areas 14. The thin layer 15 is sandwiched between the pin arrangement plate 21 and the positioning member 44, and pushed in the through hole 12 by the pins 22. Thus the top portion of the pin 22 enters the through holes 12. Thus almost all of a light path is occluded, and a light R does not enter the neighboring spot areas 14. Therefore the light seepage is prevented.

Operations of the above structure will be described in the followings. In the spotting process, the pin arrangement plate 21 presses the biochemical analysis unit 10 from the rear face. The pins 22 enter the corresponding through holes 12, and push the corresponding spot areas 14 through the thin layer 15. In this situation, the spotting is made. Since the pores 23 are squashed in the pressing, the spotted probe solution does not penetrate into the neighboring spot areas 14. Thus the labeling materials don't remain in the thin layer 15 between the neighboring spot areas. Therefore the light seepage is prevented.

In the reaction process, the biochemical analysis unit 10 is set to the reaction vessel 32, and the reaction process is performed in the procedure shown in FIG. 6. If the specific binding is made, the labeling materials remain. After the reaction process is completed, the biochemical analysis unit 10 is removed and sent to the data reading process.

In the data reading process, as shown in FIG. 6, the pin arrangement plate 21 is pressed to the rear face of the biochemical analysis unit 10 such that the pins 22 may push the corresponding spot areas 14 through the thin layer 15. In this situation, the end of the light guide 43 is confronted to the flow-through area 17, and the detection is made to obtain the result of the reaction in each spot area 14. Since each pin 22 functions as a light shielding member, the light that enters from the neighboring spot areas 14 is extremely reduced. Thus the analysis data of high accuracy is obtained.

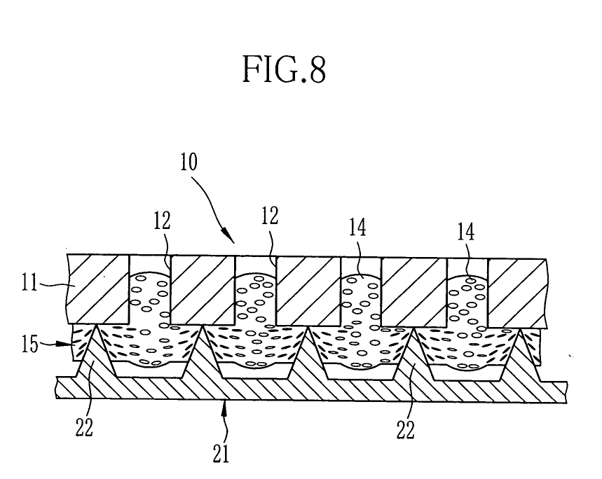

In the above embodiment, the pins press the corresponding spot areas. However, as shown in FIG. 8, the pins may push the thin layer 15 between the spot areas 14 (or between the through holes 12). Also in this case, the spotted probe solution does not penetrate into the neighboring spot areas, and the light seepage is prevented. Further, as shown in FIG. 8, the top of the pin 22 may be sharp.

In the above embodiment, the indirect labeling method in which the reaction solution with target contains no labeling materials. However the direct labeling method may be applied, in which the reaction solution contains both of the target and the labeling materials.

Various changes and modifications are possible in the present invention and may be understood to be within the present invention.

1. A spotting method for fixing a probe in plural spot areas formed in a biochemical analysis unit, said biochemical analysis unit including a substrate in which plural through holes are arranged at a predetermined pitch and a membrane which has adsorption properties and is disposed on a back of said substrate, said each spot area being constructed of said through holes and a part of said membrane charged into said through holes, said spotting method comprising steps of:
pressing plural pins on a back of said membrane, said plural pins being disposed at said predetermined pitch corresponding to said plural through holes; and
spotting a liquid of said probe into said spot areas with a spot head from an opposite side to said pin.

2. A spotting method claimed in claim 1, wherein said pin is a conic shape or a pyramidal shape, and a top of said pin is flat or sharp.

3. A spotting method claimed in claim 2, wherein said spot head contacts to said membrane through said through holes of said spot areas in said spotting step.

4. A spotting method claimed in claim 2, wherein a flange is attached to said spot head, and said flange covers said through holes in said spotting step.

5. A spotting method claimed in claim 2, wherein said each pin is positioned to said corresponding through hole, and said top of said each pin is pressed into said through hole for preventing a penetration of said probe to an outside of said through hole.

6. A spotting method claimed in claim 2, wherein said each pin is disposed between said neighboring two through holes, and said each pin pushes said membrane for preventing a penetration of said probe to an outside of said through hole.

7. A data reading method for reading a reaction result in plural spot areas formed in a biochemical analysis unit, said biochemical analysis unit including a substrate in which plural through holes are arranged at a predetermined pitch and a membrane which has adsorption properties and is disposed on a back of said substrate, said each spot area being constructed of said through holes and a part of said membrane charged into said through holes, a probe being previously fixed in said each spot area, said data reading method comprising steps of:
making a reaction solution pass through said spot area, said reaction solution containing a target as a test body, said target making a specific binding to said probe;
pressing plural pins on a back of said membrane, said plural pins being disposed at said predetermined pitch corresponding to said plural through holes; and
optically reading said reaction result in each spot area from an opposite side to said pin.

8. A data reading method claimed in claim 7, wherein said pin is a conic shape or a pyramidal shape, and a top of said pin 15 is flat or sharp.

9. A data reading method claimed in claim 8, wherein said each pin is positioned to said corresponding through hole, and said top of said each pin is pressed into said through hole for preventing an entering of a light from said neighboring spot areas.

10. A data reading method claimed in claim 8, wherein said each pin is disposed between said neighboring two through holes, and said each pin pushes said membrane for preventing an entering of a light from said neighboring spot areas.

11. A data reading method claimed in claim 8, wherein an optical fiber is contacted to said each spot area from an opposite side to said each pin in said optical reading step.

12. A data reading method claimed in claim 11, wherein said plural optical fibers are fitted to a plate, and said plate contacts to said substrate in said optical reading step.
